(19) United States
(12) Patent Application Publication
(10) Pub. No.: US 2026/0083349 A1
(43) Pub. Date: Mar. 26, 2026

(54) SELF-MIXING INTERFEROMETRY-BASED HEAD-MOUNTED RESPIRATION SENSING

(71) Applicant: Apple Inc., Cupertino, CA (US)

(72) Inventors: Yongkang Gao, Sunnyvale, CA (US); Xinsheng Chu, Saratoga, CA (US); Tong Chen, Fremont, CA (US); David D. Dashevsky, San Jose, CA (US); Javier Mendez, San Jose (UA)

(21) Appl. No.: 19/270,103

(22) Filed: Jul. 15, 2025

Related U.S. Application Data
(60) Provisional application No. 63/699,109, filed on Sep. 25, 2024.

(51) Int. Cl. A61B 5/08; A61B 5/00

(52) U.S. Cl. CPC A61B 5/0816; A61B 5/0082; A61B 5/6803; A61B 5/7445; A61B 2562/0242

(57) ABSTRACT

A device includes a head-mountable frame, an optical sensor subsystem mounted to the head-mountable frame and including a set of one or more SMI sensors, and a processor. Each SMI sensor is positioned to be spaced a respective distance from a skin surface of a user. The set of one or more SMI sensors is configured to emit a set of one or more beams of light. The processor is configured to operate the optical sensor subsystem to cause the set of one or more SMI sensors to emit the set of one or more beams of light toward the skin surface of the user; to receive a set of one or more SMI signals from the set of one or more SMI sensors; and to determine a respiration characteristic of the user based at least in part on the set of one or more SMI signals.

### CROSS-REFERENCE TO RELATED APPLICATIONS

This application claims the benefit under 35 U.S.C. § 119 (e) of U.S. Provisional Patent Application No. 63/699,109, filed Sep. 25, 2024, the contents of which are incorporated herein by reference as if fully disclosed herein.

### FIELD

The described embodiments generally relate to optical sensing and, more particularly, to respiration sensing using optical sensors.

### BACKGROUND

Respiration of a user can be monitored by audio, video, thermal, or optical means. However, ambient noise can make audio monitoring of respiration difficult. Video monitoring of respiration can be expensive, and can require significant processing resources to temporarily store and then analyze a large quantity of video information. Both ambient and body temperature changes can make thermal monitoring of respiration difficult. Optical monitoring of respiration, such as monitoring by means of a photoplethysmogram (PPG) sensor, can be useful, but requires direct contact with a user's skin. The direct skin contact can cause discomfort for some users; and in some application, direct skin contact is not possible.

### SUMMARY

Embodiments of the systems, devices, methods, and apparatus described in the present disclosure utilize one or more self-mixing interferometry (SMI) sensors to determine a respiration characteristic.

In a first aspect, the present disclosure describes a device. The device may include a head-mountable frame, an optical sensor subsystem, and a processor. The optical sensor subsystem may be mounted to the head-mountable frame and may include a set of one or more SMI sensors. Each SMI sensor of the set of one or more SMI sensors may be positioned on the head-mountable frame, to be spaced a respective distance from a skin surface of a user when the head-mountable frame is worn by the user. The set of one or more SMI sensors may be configured to emit a set of one or more beams of light that at least partially reflects from human skin. The processor may be configured to operate the optical sensor subsystem to cause the set of one or more SMI sensors to emit the set of one or more beams of light toward the skin surface of the user; to receive from the set of one or more SMI sensors, in response to the emission of the set of one or more beams of light, a set of one or more SMI signals; and to determine, based at least in part on the set of one or more SMI signals, a respiration characteristic of the user.

In a second aspect, the present disclosure describes a method for determining a respiration characteristic. The method may include operating an optical sensor subsystem to cause a set of one or more non-contact SMI sensors in the optical sensor subsystem to emit a set of one or more beams of light toward a skin surface of a user; receiving a set of one or more SMI signals from the set of one or more non-contact SMI sensors; performing at least one integration of each of the received one or more SMI signals to generate a set of integrated SMI signals; and determining a respiration characteristic based on a subset of one or more integrated SMI signals of the set of one or more integrated SMI signals.

In a third aspect, the present disclosure describes a device. The device may include a head-mountable frame, an optical sensor subsystem, and a processor. The optical sensor subsystem may be mounted to the head-mountable frame, and may include a set of one or more SMI sensors. Each SMI sensor of the set of one or more SMI sensors may be positioned on the head-mountable frame to be spaced a respective distance from a skin surface of a user when the head-mountable frame is worn by the user. The optical sensor subsystem may also include a collimator positioned in an optical path of at least one SMI sensor in the set of one or more SMI sensors. The processor may be configured to operate the optical sensor subsystem to obtain a set of one or more SMI signals from the set of one or more SMI sensors, and determine a respiration characteristic of the user based at least in part on the set of one or more SMI signals.

In addition to the exemplary aspects and embodiments described above, further aspects and embodiments will become apparent by reference to the drawings and by study of the following description.

### BRIEF DESCRIPTION OF THE DRAWINGS

The disclosure will be readily understood by the following detailed description in conjunction with the accompanying drawings, wherein like reference numerals designate like structural elements, and in which:

FIG. 1 shows an example block diagram of a respiration sensing device;

FIG. 2A shows a first example respiration sensing device in which an optical sensing subsystem and processor are mounted to a headset;

FIG. 2B shows a second example respiration sensing device in which an optical sensing subsystem and processor are mounted to a glasses frame;

FIG. 3 shows an example of an SMI sensor, in combination with an optical component, that may be used for respiration sensing;

FIG. 4 shows an example portion of a set of SMI sensors that may be mounted to a head-mountable frame and positioned to emit light toward a nose;

FIG. 5 shows an example SMI sensor, in combination with a beam steering component, that may be mounted to a head-mountable frame and positioned to scan a portion of a nose;

FIG. 6 shows an example set of SMI sensors that may be mounted to a head-mountable frame and positioned to emit light toward a portion of a skin surface adjacent an upper lip of a user; and

FIG. 7 illustrates an example method for determining a respiration characteristic of a user using a set of one or more SMI sensors.

The use of cross-hatching or shading in the accompanying figures is generally provided to clarify the boundaries between adjacent elements and also to facilitate legibility of the figures. Accordingly, neither the presence nor the absence of cross-hatching or shading conveys or indicates any preference or requirement for particular materials, material properties, element proportions, element dimensions, commonalities of similarly illustrated elements, or any other characteristic, attribute, or property for any element illustrated in the accompanying figures.

Additionally, it should be understood that the proportions and dimensions (either relative or absolute) of the various features and elements (and collections and groupings thereof) and the boundaries, separations, and positional relationships presented therebetween, are provided in the accompanying figures merely to facilitate an understanding of the various embodiments described herein and, accordingly, may not necessarily be presented or illustrated to scale, and are not intended to indicate any preference or requirement for an illustrated embodiment to the exclusion of embodiments described with reference thereto.

### DETAILED DESCRIPTION

Reference will now be made in detail to representative embodiments illustrated in the accompanying drawings. It should be understood that the following description is not intended to limit the embodiments to one preferred embodiment. To the contrary, it is intended to cover alternatives, modifications, and equivalents as can be included within the spirit and scope of the described embodiments as defined by the appended claims.

Respiration sensing provides valuable health data that may help users better understand aspects of their health and wellness. Many devices that perform respiration sensing include photoplethysmography (PPG) sensors, video imaging or similar sensors (e.g., complimentary metal-oxide semiconductor (CMOS) or similar types of imaging sensors), electromechanical-based sensors (such as a chest-strap based system), or a combination of these or other sensing technologies. However, these types of sensing approaches may present limitations. For example, video or other types of imaging-based approaches may suffer from relatively high power consumption and may require higher-performance processing capabilities in order to process larger amounts of data generated by an imaging-based sensor. As another example, PPG-based devices require contact between each PPG sensor and the skin of the user in order to acquire a respiration signal. In some use-cases, having a sensor worn against the skin may cause user discomfort or may result in other user limitations. Further, PPG-based sensors may be subject to motion artifacts caused by movements of the user (e.g., walking, postural changes, etc.), which may corrupt an acquired respiration signal.

The following description relates to non-contact respiration sensing using SMI sensors. For purposes of this description, an SMI sensor is considered to include a light emitter (e.g., a laser light source) and an SMI signal detector (e.g., a light detector, such as a photodiode, or an electrical detector, such as a circuit that measures the junction voltage or drive current of a light emitter). Each of one or more SMI sensors may emit one or more fixed or scanned beams of light toward one or more portions of a user's face (e.g., toward one or more of portions of the user's nose, toward areas adjacent the lips and/or mouth, or toward other areas of the face or head). The SMI sensor(s) may be operated in compliance with operational safety specifications, so as to not harm the user.

After emitting a beam of light, an SMI sensor may receive a retroreflected portion of the emitted light (e.g., part of a reflection or backscatter) back into its resonant cavity. The phase change of the retroreflected portion of the emitted light may mix with the phase of the light that is generated within the resonant cavity, and may produce an SMI signal that can be amplified and detected. The amplified SMI signal, and in some cases multiple SMI signals, may be analyzed to determine a time-varying displacement of a user's skin surface, which time-varying displacement can be used to determine a respiration characteristic of the user.

An SMI sensor oriented perpendicular to a skin surface may maximize or improve the output signal quality of the SMI signal, by improving the amount of retroreflected light received by the SMI sensor from the skin surface. SMI sensors oriented at a non-perpendicular angle from a surface may receive a retroreflected portion of the emitted light, but the output SMI signal may be weaker or of lower quality relative to an SMI signal of an SMI sensor oriented perpendicular to the skin surface. As used herein, the term “signal quality” may refer to a measure of the amplitude of an SMI signal (e.g., peak amplitude, peak-to-peak amplitude, etc.), signal-to-noise ratio (SNR), and/or other measures of signal quality of an SMI signal.

When the wavelength of the beam of light emitted by an SMI sensor is modulated, an SMI signal obtained from the SMI sensor may be used for relative or absolute ranging of surfaces of a user's nose or other portions of the user's head or face, with a level of resolution on the order of ˜100 μm. Such relative or absolute distance measurements can be used to determine a respiration characteristic of a user.

During respiration, airflow through portions of the upper respiration system (e.g., through the nasal passages and/or the mouth), may cause skin surface displacement associated with the airflow. For instance, skin surfaces on or around the nose may be displaced as the volume of air within the nose increases and/or decreases during respiration. In one example, an exhalation may cause a skin surface of the nose to be displaced outward from the nose (e.g., an expansion of the volume of air within the nose) and an inhalation may cause the skin surface of the nose to be displaced inward toward the nose (e.g., a contraction of the volume of air within the nose). In another example, an exhalation may cause minimal displacement of the skin surface of the nose, but an inhalation may cause measurable displacement. In other examples, respiration may cause varying degrees of skin surface displacement of portions of the nose, areas adjacent the lips and/or mouth, and/or other portions of the face or head. In some cases, the displacements associated with respiration (expansion or contraction of a volume of air within the nose or mouth) may be on the order of hundreds of micrometers, or may be larger or smaller. One or more SMI sensors that are configured to direct emitted light toward such a skin surface may provide one or more SMI signals from which skin surface displacements associated with a user's respiration and/or a respiration characteristic of the user can be determined. In some instances, an SMI signal may be processed to determine a respiration characteristic. For instance, signal filtering may be applied to the SMI signal, and/or the SMI signal may be processed through a Fast Fourier Transform (FFT) algorithm, a peak-finding algorithm, and/or through other types of algorithms (or otherwise processed) to determine a respiration characteristic of a user. For example, the SMI signal may be processed to determine a signal representative of skin surface velocity, which may correspond to user respiration. In other examples, the velocity signal (or other signal) may be mathematically integrated to provide a skin surface displacement signal that corresponds to user respiration (e.g., a time-varying change in respiration flow). In still other examples, the skin surface displacement signal may itself be integrated to provide a signal representative of respiration (e.g., a time-varying change in respiration volume). As used herein, the term “respiration signal” may indicate an SMI-based signal or processed SMI-based signal that represents a time-varying skin surface velocity, a time-varying skin surface displacement, a time-varying respiration volume, and/or other signals that correspond to one or more aspects of respiration. As also used herein, the term “respiration characteristic” may refer to a respiration rate, a respiration flow, a respiration volume, information about regular or irregular breathing (e.g., breathing pattern disorder (BPD), dyspnea, orthopnea, apnea, Cheyne-Stokes respiration, bradypnea, tachypnea, hyperpnea, hyperventilation, hypoventilation, asthma, etc.), an estimation of respiration quality, and so on.

As described herein, in some embodiments, one or more SMI sensors may direct light towards portions of the nose, areas adjacent the lips and/or mouth, etc. As such, one or more of the SMI sensors may provide SMI signals associated with respiration that is predominantly through the nose (e.g., nasal respiration), predominantly through the mouth (e.g., oral respiration), or a combination of nasal and oral respiration. The corresponding SMI signals may be processed to determine a respiration characteristic as described herein.

Devices described herein include head-mounted devices (e.g., headsets, goggles, eyeglasses, etc.) that are integrated with (e.g., include as part of the head-mounted device) or associated with (e.g., as a remote module) a processor that process one or more SMI signals to determine a further respiration signal and/or respiration characteristic of a user. For example, the device may determine a respiration rate, or may estimate a respiration (breathing) volume, based on an SMI or respiration signal. In further examples, the respiration signal may be used to detect regular or irregular breathing patterns or conditions, such as BPD, dyspnea, orthopnea, apnea, Cheyne-Stokes respiration, bradypnea, tachypnea, hyperpnea, hyperventilation, hypoventilation, asthma, etc.). In some embodiments, the device may use the respiration signal as part of a wellness feature, such as for controlled breathing exercises, for determining a level of nasal congestion, and/or for other health or wellness features.

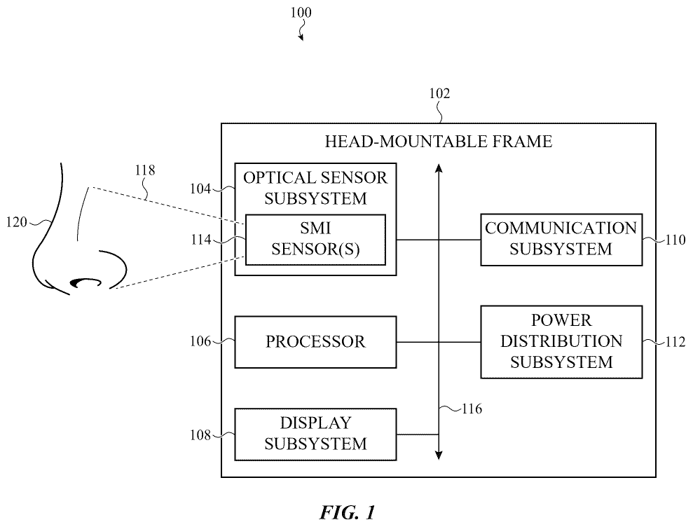

These and other systems, devices, methods, and apparatus are described with reference to FIGS. 1-6. However, those skilled in the art will readily appreciate that the detailed description given herein with respect to these figures is for explanatory purposes only and should not be construed as limiting.

Directional terminology, such as “top”, “bottom”, “upper”, “lower”, “front”, “back”, “over”, “under”, “above”, “below”, “left”, “right”, etc. may be used with reference to the orientation of some of the components in some of the figures described below. Because components in various embodiments can be positioned in a number of different orientations, directional terminology is used for purposes of illustration only and is not always limiting. The directional terminology is intended to be construed broadly, and therefore should not be interpreted to preclude components being oriented in different ways. Also, as used herein, the phrase “at least one of” preceding a series of items, with the term “and” or “or” to separate any of the items, modifies the list as a whole, rather than each member of the list. The phrase “at least one of” does not require selection of at least one of each item listed; rather, the phrase allows a meaning that includes at a minimum one of any of the items, and/or at a minimum one of any combination of the items, and/or at a minimum one of each of the items. By way of example, the phrases “at least one of A, B, and C” or “at least one of A, B, or C” each refer to only A, only B, or only C; any combination of A, B, and C; and/or one or more of each of A, B, and C. Similarly, it may be appreciated that an order of elements presented for a conjunctive or disjunctive list provided herein should not be construed as limiting the disclosure to only that order provided.

FIG. 1 shows an example block diagram of a respiration sensing device 100. The respiration sensing device 100 may include a head-mountable frame 102, an optical sensor subsystem 104 mounted to the head-mountable frame 102, and a processor 106. In some embodiments, the respiration sensing device 100 may also include one or more of a display subsystem 108, a communication subsystem 110 (e.g., a wireless and/or wired communication subsystem), and a power distribution subsystem 112. The processor 106, display subsystem 108, communication subsystem 110, and power distribution subsystem 112 may be partly or wholly mounted to the head-mountable frame 102, or in radio frequency or electrical communication with one or more components mounted to the head-mountable frame 102 (e.g., housed in a box or electronic device (e.g., a phone, or a wearable device such as a watch) that is in radio frequency (e.g., wireless) or electrical (e.g., corded) communication with one or more components mounted to the head-mountable frame 102), or distributed between the head-mountable frame 102 and a box or electronic device that is in radio frequency or electrical communication with one or more components mounted to the head-mountable frame 102. The optical sensor subsystem 104, processor 106, display subsystem 108, communication subsystem 110, and/or power distribution subsystem 112 may communicate over one or more buses 116, or over the air (e.g., wirelessly), using one or more communication protocols.

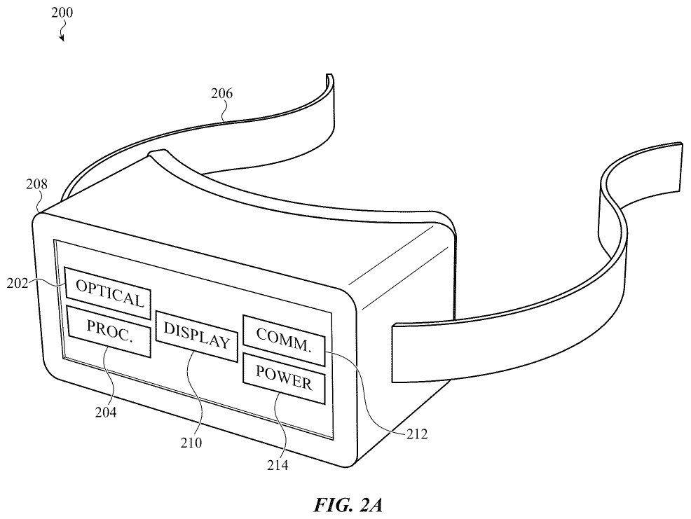

The head-mountable frame 102 may take the form of a glasses frame, goggles, a headset (e.g., an augmented reality (AR) headset or a virtual reality (VR) headset), or other form of head-mountable frame 102. Example head-mountable frames are depicted in FIGS. 2A and 2B.

The optical sensor subsystem 104 may include a set of one or more SMI sensors 114. Each SMI sensor in the set of one more SMI sensors may include a light emitter and a light detector. The light emitter may include one or more of a vertical-cavity surface-emitting laser (VCSEL), an edge-emitting laser (EEL), a vertical external-cavity surface-emitting laser (VECSEL), a quantum-dot laser (QDL), a quantum cascade laser (QCL), or a light-emitting diode (LED) (e.g., an organic LED (OLED), a resonant-cavity LED (RC-LED), a micro LED (mLED), a superluminescent LED (SLED), or an edge-emitting LED), and so on. The light detector (or photodetector) may in some cases be positioned laterally adjacent the light emitter (e.g., mounted or formed on a substrate on which the light detector is mounted or formed). In other cases, the light detector may be stacked above or below the light emitter. For example, the light detector may be a VCSEL, a horizontal cavity surface-emitting laser (HCSEL), or EEL having a primary emission and a secondary emission, and the light detector may be epitaxially formed in the same epitaxial stack as the light emitter, such that the light detector receives some or all of the secondary emission. In these latter embodiments, the light emitter and light detector may be similarly formed (e.g., both the light emitter and the light detector may include multiple quantum well (MQW) structures, but the light emitter may be forward-biased and the light detector (e.g., a resonant cavity photodetector (RCPD) may be reverse-biased). Alternatively, the light detector may be formed on a substrate and the light emitter may be separately formed and mounted to the substrate, or in relation to the light emitter, such that a secondary light emission of the light emitter impinges on the light detector. Alternatively, the light emitter may be formed on a substrate and the light detector may be separately formed and mounted to the substrate, or in relation to the light emitter, such that secondary light emission of the light emitter impinges on the light detector.

In some embodiments, the optical sensor subsystem 104 may include a set of one or more stationary or movable optical components (e.g., one or more lenses, optical gratings, optical filters, beam splitters, beam steering components, collimators, and so on). Some or all of the optical components may be positioned in an optical path (or optical paths) of one or more of the SMI sensor(s) 114. The optical sensor subsystem 104 may also include an image sensor (e.g., a camera including an image sensor).

The processor 106 may include any electronic device capable of processing, receiving, or transmitting data or instructions, whether such data or instructions are in the form of software or firmware or otherwise encoded. For example, the processor 106 may include a microprocessor, a central processing unit (CPU), a graphics processing unit (GPU), an application-specific integrated circuit (ASIC), a digital signal processor (DSP), a controller, or a combination of such devices. As described herein, the term “processor” is meant to encompass a single processor or processing unit, multiple processors or processing units, or other suitably configured computing elements.

The components of the respiration sensing device 100 can be controlled by multiple processors in some embodiments. For example, select components of the respiration sensing device 100 (e.g., components of the optical sensor subsystem 104) may be controlled by a first processor, and other components of the respiration sensing device 100 (e.g., the display subsystem 108 and/or communication subsystem 110) may be controlled by a second processor, where the first and second processors may or may not be in communication with each other.

In some embodiments, the display subsystem 108 may include a display having one or more light-emitting elements including, for example, LEDs, OLEDs, a liquid crystal display (LCD), an electroluminescent (EL) display, or other types of display elements. In some embodiments, the display subsystem 108 may include a heads up display (HUD).

The communication subsystem 110 may enable the respiration sensing device 100 to transmit to, or receive data from, a user or another electronic device. The communication subsystem 110 may include a touch sensing input surface, a crown, one or more microphones or speakers, or a wired or wireless (e.g., radio frequency (RF) or optical) communications interface configured to transmit electronic, RF, or optical signals. Examples of wireless and wired communications interfaces include, but are not limited to, cellular, Wi-Fi, and BLUETOOTH® communications interfaces.

The power distribution subsystem 112 may be implemented with any collection of power sources and/or conductors capable of delivering energy to the respiration sensing device 100 or components thereof. In some cases, the power distribution subsystem 112 may include one or more batteries or rechargeable batteries. Additionally or alternatively, the power distribution subsystem 112 may include a power connector, charging interface, or power cord that can be used to connect the respiration sensing device 100 or its components to a remote power source, such as a wall outlet, remote battery pack, wireless charger, or electronic device to which the respiration sensing device 100 is tethered.

The processor 106 may be configured to operate the optical sensor subsystem 104 and/or other subsystems of the respiration sensing device 100. Operating the optical sensor subsystem 104 may include causing the power distribution subsystem 112 to power the optical sensor subsystem 104, providing control signals to the set of one or more SMI sensors 114, and/or providing control signals that electrically, electromechanically, or otherwise focus or adjust optical components (e.g., lenses, lens assemblies, beam steering components, etc.) of the optical sensor subsystem 104. Operating the optical sensor subsystem 104 may cause the set of one or more SMI sensors 114 to emit a set of one or more beams of light 118 toward a portion of the user's head and/or face, such as portions of a nose 120 of a user, or another portion of the user's head and/or face (e.g., portions of the upper lip, sides of the mouth, etc.). The processor 106 may also be configured to receive a set of one or more SMI signals from the set of one or more SMI sensors 114 and process the SMI signals to determine and track skin surface velocity, skin surface displacement, respiration flow, respiration volume, and/or other respiration characteristics.

In some cases, the optical sensor subsystem 104, processor 106, display subsystem 108, communication subsystem 110, and/or power distribution subsystem 112 may communicate over one or more busses, which are generally portrayed as bus 116.

In some embodiments, the processor 106 may be configured to receive, determine, and/or analyze the respiration signal and identify respiration events or conditions, or other aspects of respiration, based on the respiration signal analysis. For example, the processor 106 may analyze the respiration signal and identify portions of the respiration signal associated with asthma. In other examples, the processor 106 may identify nasal congestion and/or other respiration conditions based on analysis of the respiration signal. In other examples, the processor 106 may identify other events, conditions, or regular or irregular breathing patterns that may be determined from the respiration signal (e.g., any of the regular or irregular breathing patterns or conditions described herein). In some instances, the processor 106 may provide an alarm, an alert, a message, and/or other indication of an identified respiration event or condition, such as through the display subsystem 108 and/or the communication subsystem 110.

FIG. 2A shows a first example respiration sensing device in which an optical sensing subsystem 202 and processor 204 are mounted to a headset (e.g., a virtual reality (VR) headset 200 or an augmented reality (AR) headset). By way of example, the headset 200 (e.g., a type of head-mountable frame) is shown to include a module 208 that can be attached to a user's head by a strap 206. The module 208 may include a display subsystem 210. The display subsystem 210 may include a display for displaying text, numbers, and/or images to a user of the headset 200.

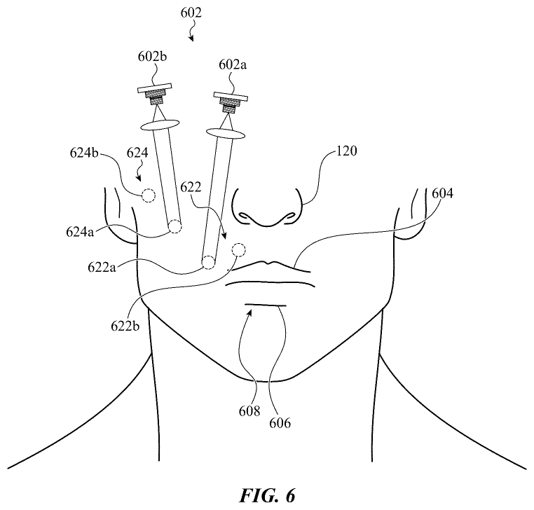

By way of example, the optical sensing subsystem 202 may be configured as described with reference to one or more of FIGS. 1 and 3-5, and/or the processor 204 may be configured to operate as described with reference to one or both of FIGS. 1 and 6. One or more components (e.g., SMI sensors, optical components, and so on) of the optical sensing subsystem 202 may be mounted on one or more substrates attached to or housed within the module 208, or may be mounted directly to a housing of the module 208. Similarly, the processor 204, display subsystem 210, a communication subsystem 212, and/or a power distribution subsystem 214 may be mounted to the module 208. In some embodiments, part or all of the optical sensing subsystem 202, processor 204, display subsystem 210, communication subsystem 212, and/or power distribution subsystem 214 may be mounted within a device that is wirelessly or electrically connected to the module 208 (e.g., in a user's phone or wearable device), or distributed between the module 208 and the device that is wirelessly or electrically connected to the module 208.

The processor 204, display subsystem 210, communication subsystem 212, and power distribution subsystem 214 may be further configured or operated as described with reference to FIG. 1. For instance, when the headset 200 is worn by the user, the processor 204 may be configured to operate the optical sensing subsystem 202, to cause a set of one or more SMI sensors to emit a set of one or more beams of light toward the skin surface of the nose, areas surrounding the mouth, etc., as described herein.

FIG. 2B shows a second example respiration sensing device in which an optical sensing subsystem 252 and processor 254 are mounted to a glasses frame 250. By way of example, the glasses frame 250 (e.g., a type of head-mountable frame) is shown to include a first lens rim 258 and a second lens rim 260, a bridge 262 connecting the first lens rim 258 to the second lens rim 260, a first temple 264 connected to the first lens rim 258, and a second temple 266 connected to the second lens rim 260. In some embodiments, the glasses frame 250 may include a heads up display or function as AR glasses.

Each of the first lens rim 258 and the second lens rim 260 may hold a respective lens, such as a first lens 268 or a second lens 270. The lenses 268, 270 may or may not magnify, focus, or otherwise alter light passing through the lenses 268, 270. For example, the lenses 268, 270 may correct a user's vision, block bright or harmful light, or simply provide a physical barrier through which light can pass with no or minimal adjustment. In some embodiments, the first and second lenses 268, 270 may be formed of glass or plastic. In some embodiments, the first and/or second lenses 268, 270 may function as a display (e.g., a passive display screen) on which text, numbers, and/or images are projected by a display subsystem 272, which display subsystem 272 may also be mounted to the glasses frame 250. Alternatively, the first and/or second lens rims 258, 260 may hold a transparent or translucent display (e.g., light-emitting diodes (LEDs), organic LEDs (OLEDs), or other light-emitting elements that can be operated by the display subsystem 272 to display text, numbers, and/or images.

By way of further example, the optical sensing subsystem 252 may be configured as described with reference to one or more of FIGS. 1 and 3-5, and/or the processor 254 may be configured to operate as described with reference to one or both of FIGS. 1 and 6. Although the optical sensing subsystem 252 is depicted in FIG. 2B as mounted to or within the second lens rim 260, one or more components (e.g., SMI sensors, optical components, and so on) of the optical sensing subsystem 252 may be mounted on one or more substrates attached to, the first lens rim 258, second lens rim 260, bridge 262, first temple 264, second temple 266, first lens 268, or second lens 270, or may be mounted directly to one or more of these components. Similarly, the processor 254, display subsystem 272, a communication subsystem 274, and/or a power distribution subsystem 276 may be mounted directly or indirectly, on or within, one or more of these components. In some embodiments, part or all of the optical sensing subsystem 252, processor 254, display subsystem 272, communication subsystem 274, and/or power distribution subsystem 276 may be mounted within one or more components of the glasses frame 250, within a device that is wirelessly or electrically connected to one or more components mounted to the glasses frame 250 (e.g., in a user's phone or wearable device), or distributed between the glasses frame 250 and a device that is wirelessly or electrically connected to one or more components of the glasses frame 250.

The processor 254, display subsystem 272, communication subsystem 274, and power distribution subsystem 276 may be further configured or operated as described with reference to FIG. 1. For instance, when the glasses frame 250 is worn by the user, the processor 254 may be configured to operate the optical sensing subsystem 252 to cause a set of one or more SMI sensors to emit a set of one or more beams of light 278 toward the skin surface of the nose, areas surrounding the mouth, etc., as described herein.

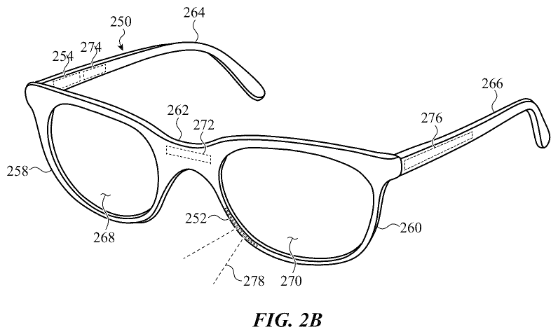

FIG. 3 shows an example of an SMI sensor 302, in combination with an optical component 306, that may be used for respiration sensing. The SMI sensor 302 may be any of the SMI sensors described herein. The optical component 306 may include one or multiple elements that, together, define the optical component 306 and its function(s).

The SMI sensor 302 and optical component 306 may be mounted to a head-mountable frame, such as one of the head-mountable frames depicted in FIG. 2A or 2B and described herein (or as part of another type of head-mounted device). The SMI sensor 302 may be positioned on a head-mountable frame such that it will be spaced a respective distance from a skin surface of a user when the head-mountable frame is worn by the user. The respective distance between the SMI sensor 302 and the skin surface of a user will typically not be an absolute distance, and may vary depending on whether the head-mountable frame is worn by User A or User B, and on how the user wears the head-mountable frame (e.g., loosely or tightly, or resting high or low on their nose bridge). The respective distance may be a target or estimated distance, or may represent a working range of distances.

The optical component 306 may be positioned a distance D₁ from the SMI sensor 302 and configured to modify a beam of light 304 emitted by the SMI sensor 302. In some variations, the distance D₁ may be less than 1 mm. In other variations, the distance D₁ may be approximately 1 mm or more than 1 mm. Distance D₁ may be selected based on one or more desired optical performance criteria, such as to achieve a desired degree of collimation, or for another purpose.

In the example depicted in FIG. 3, the optical component 306 may be a collimator. The collimator may receive the beam of light 304, collimate the light, and direct the resulting collimated beam of light 308 away from the SMI sensor 302, such as toward a skin surface 310. The SMI sensor 302 and optical component 306 may be mounted to the head-mountable frame such that when the device is being worn by a user, the optical component 306 is positioned a distance D₂ from the skin surface 310. As may be appreciated, the distance D₂ may be different for different users, may be different for the same user over different uses of the device, or may vary over the course of a single use of the device. Typically, the SMI sensor 302 will be positioned on the head-mountable frame such that the distance D₂ is likely to fall within a working range of distances regardless of which user wears the head-mountable frame and regardless of how a user wears the head-mountable frame (within reason).

The distance D₂ may be a distance at which the SMI sensor 302 can adequately receive at least part of a reflection or backscatter (e.g., a retroreflection) of an emitted beam of light. The retroreflection may be a reflection from human skin (e.g., the skin surface 310). The distance D₂, or desired working range of distances in which distance D₂ falls, may be a distance that enables the SMI sensor 302 to provide an SMI signal having a signal quality that is suitable for determining a displacement of the skin surface 310 during respiration. For example, the SMI sensor 302 and optical component 306 may be mounted to the head-mountable frame such that distance D₂ is no greater than 60 mm during use. However, in some variations, the SMI sensor 302 may provide a suitable SMI signal when distance D₂ is greater than 60 mm. Preferably, distance D₂ may lie in the range of 30-40 mm. Further, the SMI sensor 302 may provide a suitable SMI signal when distance D₂ is greater than 0 mm, but less than 5 mm, 10 mm, or 15 mm.

As described herein, the optical component 306 may be a collimator that shapes a collimated beam of light 308. In some embodiments, the optical component 306 may be selected to provide a collimated beam of light 308 that converges to a minimum beam width of W₁ at a distance D₃ from the optical component 306, and to diverge thereafter. The minimum beam width W₁ at distance D₃ may be approximately 300 μm, but in some variations may be greater than or less than 300 μm. The minimum beam width of W₁ should be substantially greater than zero, such that the collimated beam of light 308 never converges to a focal point. However, given that true collimation, at all distances, is difficult to achieve, the collimated beam of light 308 may converge and then diverge, somewhat converge, or somewhat diverge within a desired range of working distances. Stated differently, the collimated beam of light 308 may be approximately collimated within the desired range of working distances.

The distance D₃ may represent a target distance between the skin surface 310 and the optical component 306, such that when the distance D₂ between the optical component 306 and the skin surface 310 equals target distance D₃, the SMI sensor 302 may provide an output SMI signal with a relatively higher signal quality than at other distances D₂. In some embodiments, the target distance D₃ may be within 30-40 mm. In some variations, the target distance D₃ may be 5 mm, 10 mm, 15 mm, or more than 40 mm.

In cases where the distance D₂ is greater than the target distance D₃, as shown in FIG. 3, the collimated beam of light 308 may diverge and illuminate a portion of the skin surface with a beam width W₂ that is larger than W₁. In examples where distance D₂ is at a maximum preferrable range (e.g., 60 mm), the beam width W₂ may be approximately 400 μm. In other examples, the beam width W₂ may be larger or smaller than 400 μm. For infrared light, a narrower beam width (e.g., in the general range of 300-400 μm) may allow the collimated beam of light 308 to interact more predominantly with the skin surface 310, rather than penetrating the skin surface 310 and interacting predominantly with the underlying tissue. Accordingly, the SMI signal provided by the SMI sensor may include characteristics that more accurately represents displacement of the skin surface 310. In alternative examples, the beam width W₂ may be smaller than W₁, but closer to W₁ than to a focal point.

FIG. 3 further depicts an example of how respiration may be measured by the SMI sensor 302. The collimated beam of light 308 may include light traveling in direction A1, from the optical component 306 towards the skin surface 310. During respiration, air may flow in direction A2, within an interior respiration passage 312. The airflow may be associated with an increase in pressure within the respiration passage 312, such as may occur during an exhalation through a nasal passage. The increased pressure may cause the skin surface 310 to displace in direction A3. A portion of the light of the collimated beam of light 308 may retroreflect from the skin surface 310 during the displacement, and impinge on the optical component 306 from direction A4. The amount of light retroreflected from the skin surface may be affected by the angle θ of the skin surface 310 with respect to a cross-section of the collimated beam of light 308. For instance, the highest degree of retroreflection may be obtained when the skin surface 310 is perpendicular to the direction A1 of the collimated beam of light 308, or when angle θ is 0°. At other values of 0, the amount of retroreflected light may decrease relative to when angle θ is 0°. It should be appreciated that the angle θ of the skin surface 310 can depend on the beam cross-section for which the angle θ is measured.

In other examples, such as during an inhalation through a nasal passage, airflow in the respiration passage 312 may flow in the opposite of direction A2, and the skin surface 310 maybe displaced in a direction opposite of direction A3 (e.g., the skin surface 310 may contract inward).

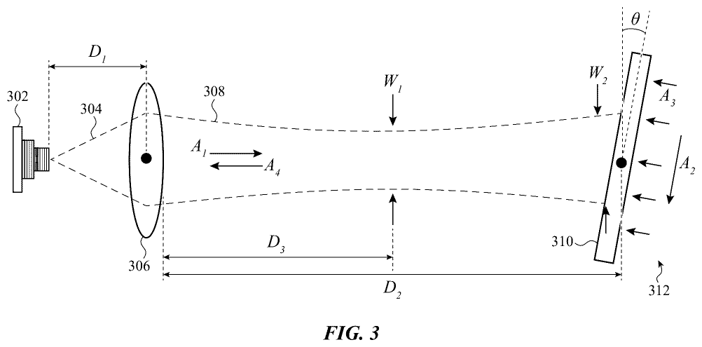

FIG. 4 shows an example portion of a set of SMI sensors 402 that may be mounted to a head-mountable frame, such as one of the head-mountable frames described with reference to FIGS. 1-2B. The set of SMI sensors 402 (one or more SMI sensors) may include any of the SMI sensors described herein. The set of SMI sensors 402 may include a first SMI sensor 402a and a second SMI sensor 402b, which may emit respective beams of light toward the nose 120. In other examples, the set of SMI sensors 402 may include more or fewer SMI sensors. The set of SMI sensors 402 may emit respective beams of light that impinge on the nose at a set of respective regions 422. For instance, the first SMI sensor 402a may emit a beam of light that impinges on the nose at a first region 422a, the second SMI sensor 402b may emit a beam of light that impinges on the nose at a second region 422b, and so on.

The regions of the set of regions 422 may experience varying levels of skin surface displacement during respiration for a given user. In one scenario, for a first user, the skin surface corresponding to the second region 422b may experience larger displacements during respiration than the skin surface corresponding to the first region 422a and other regions of the set of regions 422. Accordingly, the second SMI sensor 402b may provide an SMI signal having a higher signal quality relative to the remaining SMI sensors. For a second user, the skin surface corresponding to a third region 422c associated with a third SMI sensor (not depicted) may experience larger skin surface displacements than the other regions of the set of regions 422. Accordingly, the third SMI sensor may provide an SMI signal having a higher signal quality relative to the remaining SMI sensors. A processor that receives the SMI signals generated by the set of SMI sensors 402 may analyze the received SMI signals to determine which SMI signal (or subset of one or more SMI signals) has the highest signal quality (or more generally, a particular signal quality). The processor may select the corresponding SMI sensor(s) to provide an SMI signal (or signals) upon which a respiration signal and/or respiration characteristic may be based.

In another scenario, on a subsequent use of the head-mountable frame, the first user may wear the head-mountable frame in a different position on their head. For instance, the first user may wear the head-mountable frame higher on the face or nose 120, such that the set of SMI sensors 402 is shifted vertically from the depiction of FIG. 4. As a result, the first SMI sensor 402a may emit a beam of light that impinges on the second region 422b, or a portion of the nose 120 near the second region 422b, where the skin surface may experience greater displacement during respiration. The second SMI sensor 402b may emit a beam of light that impinges on another (higher) region of the nose 120. Accordingly, the first SMI sensor 402a may provide an SMI signal with the highest signal quality, and the respiration signal and/or respiration characteristic may be based on the SMI signal generated by the first SMI sensor 402a.

In still another scenario, during a session in which the user wears the head-mountable frame, the head-mountable frame may shift position on the user's head (e.g., as a result of user motion), causing each SMI sensor of the set of SMI sensors 402 to measure skin surface displacement on another portion of the nose 120. The processor that receives the SMI signals may analyze the SMI signals and detect a change in signal quality from one or more of the SMI sensors. For instance, the processor may determine that a first SMI sensor provides the highest signal quality. Following a shift in position of the head-mountable frame, the processor may detect that a second SMI sensor provides the highest signal quality and may use the SMI signal generated by the second SMI sensor to generate a respiration signal and/or determine a respiration characteristic.

It should be appreciated that the locations of the set of regions 422 on the nose 120 (or portions of the upper lip, etc.) may vary between different users, based on how a given user wears the head-mountable device, user anatomy (e.g., shape of the nose, mouth, face, head, etc.), fitment of the head-mountable device, and/or a range of other factors. In some variations, the set of SMI sensors 402 may be arranged such that the corresponding set of regions 422 covers a larger or smaller portion of the nose 120. For example, in some variations, the regions 422 may be spaced farther apart or more closely together. As described herein, the processor may determine which of the SMI sensors provides an SMI signal with the highest signal quality or of a particular quality, and may generate the respiration signal and/or determine a respiration characteristic based on this SMI signal (or subset of SMI signals).

In this manner, a respiration signal and/or respiration characteristic may be determined under varying use-case conditions. Furthermore, this method of determining a subset of one or more SMI sensors having the highest signal quality (or a particular quality) may allow the device to perform consistent respiration measurements over a population of users, where anatomical and other differences between users may hinder other approaches.

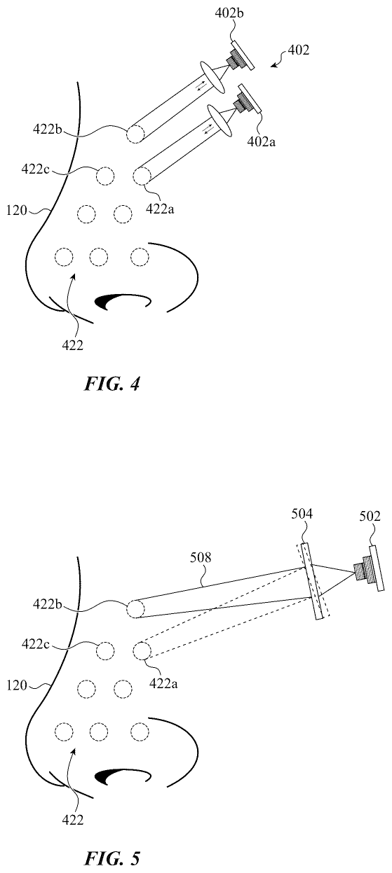

FIG. 5 shows an example SMI sensor 502, in combination with a beam steering component 504, that may be mounted to a head-mountable frame and positioned to scan a portion of a nose 120. The SMI sensor 502 may be any of the SMI sensors described herein. The beam steering component 504 may be positioned to steer a beam of light 508 emitted by the SMI sensor 502 and passed through the beam steering component 504. A processor may be configured to operate the beam steering component 504 and steer the beam of light 508 to different structures or areas of the face or head, such as the nose 120 or portions of the upper lip. In some embodiments, the beam steering component 504 may include a beam focusing component or lens positioning mechanism, which beam focusing component or lens positioning mechanism may be adjusted to change the focus of the beam along its axis. Additionally or alternatively, the beam steering component 504 may include a collimator that collimates the beam of light as described herein. Accordingly, the beam of light 508 may be a collimated beam of light.

In some cases, the processor may cause the SMI sensor 502 to scan the set of regions 422 by steering the beam of light 508 to each region, or by sweeping the beam of light 508 over a larger region including the set of regions 422. The SMI signal generated by the SMI sensor 502 for each region of the set of regions 422 may be analyzed to determine which of the regions is associated with a particular, or highest, signal quality (or determine a particular orientation of the beam of light 508, which particular orientation produces an SMI signal with a particular, or highest, signal quality). The beam steering component 504 may then orient the beam of light 508 in a particular orientation, to direct the beam of light 508 to the region associated with the highest signal quality, and a respiration signal and/or respiration characteristic may be based on the corresponding SMI signal.

In other cases, the beam steering component 504 may be operated to scan more or fewer regions of the set of regions 422, and may be operated to scan other portions of the nose, areas around the upper lip, and/or other portions of the face or head.

In some variations, after determining a region associated with the highest, or particular, signal quality, and orienting the beam steering component 504 to direct the beam of light 508 to that region, processor may subsequently operate the beam steering component to scan the set of regions 422. For example, the processor may be configured to periodically perform a scan of the set of regions 422 to determine changes in signal quality that may be associated with positional changes of the head-mountable frame. In another example, the processor may be configured to detect a reduction in signal quality associated with a particular region of the set of regions 422, and may automatically perform a scan of the set of regions 422 to identify a region with improved signal quality. In this manner, a respiration signal and/or respiration characteristic may be determined under varying use-case conditions, such that respiration measurements may be robust against various amounts of motion, different positions or fitments of the head-mountable frame, and the like. Furthermore, this method of scanning portions of the nose (or other portions of the face) to determine a location with the highest signal quality may allow the device to perform accurate respiration measurements over a population of users, where anatomical and other differences between users may hinder other approaches.

FIG. 6 shows an example portion of a set of SMI sensors 602 that may be mounted to a head-mountable frame, such as one of the head-mountable frames described with reference to FIGS. 1-2B. The set of SMI sensors 602 (one or more SMI sensors) may include any of the SMI sensors described herein. The set of SMI sensors 602 may include a first SMI sensor 602a and a second SMI sensor 602b, which may emit respective beams of light. In other examples, the set of SMI sensors 602 may include more or fewer SMI sensors.

The set of SMI sensors 602 may emit respective beams of light. At least one of the beams of light may impinge on a respective region 622a of a set of one or more regions 622 adjacent an upper lip 604 of a user (and in some cases, between the upper lip 604 and the nose 120, or on the upper lip 604, or on a lower lip 606). At least one other beam of light may impinge on a respective region 624a of a set of one or more regions 624 more distant from the upper lip 604 (and more distant from a mouth 608 bounded by the upper and lower lips 604, 606). For instance, the first SMI sensor 602a may emit a beam of light that impinges on a first region 622a adjacent the upper lip 604, and the second SMI sensor 602b may emit a beam of light that impinges on a second region 624a, the second region 624a being on the user's check or check bone, and so on. The regions 624a, 624b in the second set of one or more regions 624 may be selected based on their being relatively unaffected by a user's respiration activity, and based on the first and second sets of one or more regions 622, 624 being relatively similarly affected by sound waves, ambient air movement, and so on. In this manner, and by way of example, an SMI signal obtained from the second SMI sensor 602b may be used by a processor or other circuitry as a reference signal, to cancel common mode noise (e.g., non-respiration affects) from an SMI signal obtained from the first SMI sensor 602a.

In alternative embodiments of what is shown in FIG. 6, the set of SMI sensors 602 may be replaced by a singular SMI sensor that is configured to scan a single beam of light across each of the regions 622 and 624 (e.g., as described with reference to FIG. 5). Alternatively, the first SMI sensor 602a may scan a beam of light across the region 622, including regions 622a and 622b, and the second SMI sensor 602b may scan beam of light across the region 624, including regions 624a and 624b.

In some embodiments, the systems described with reference to FIGS. 4 and 5 may be adapted to include one or more SMI sensors having beams of light that are directed toward a user's cheek, cheek bone, or other facial area that is relatively unaffected by a user's respiration activity, but affected by sound waves, ambient air movement, and so on relatively similarly to the nose 120. In this manner, an SMI signal obtained from such an SMI sensor may be used by a processor or other circuitry as a reference signal, to cancel common mode noise (e.g., non-respiration affects) from an SMI signal obtained from the SMI sensors 402 (FIG. 4) or SMI sensor 502 (FIG. 5).

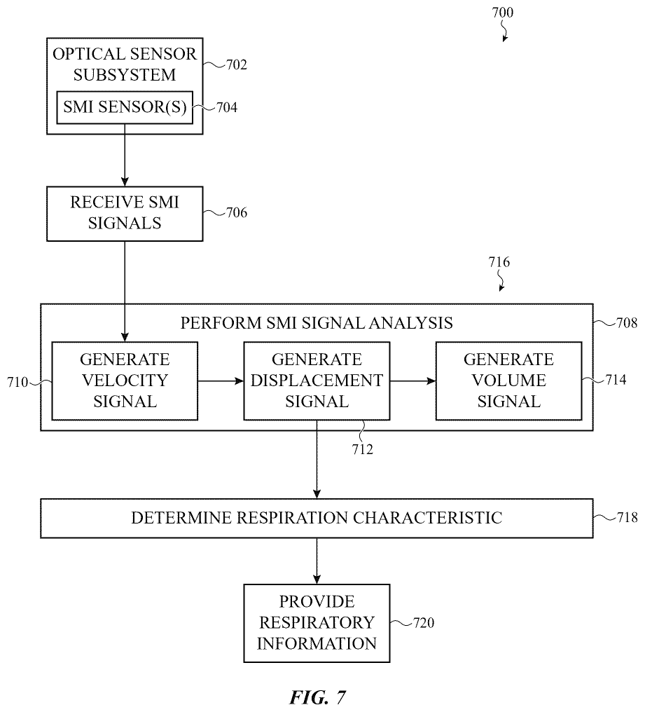

FIG. 7 illustrates an example method for determining a respiration characteristic of a user using a set of one or more SMI sensors 704. The one or more SMI sensors 704 may be non-contact SMI sensors (e.g., SMI sensors that are each positioned a respective distance from a skin surface of a user). The method 700 may include operating an optical sensor subsystem 702 to cause the set of one or more SMI sensors 704 to emit a set of one or more beams of light toward a skin surface of a user, such as a skin surface on a side of the user's nose or a skin surface adjacent the user's upper lip. The optical sensor subsystem 702 and SMI sensor(s) 704 may be configured similarly to any of the optical sensor subsystems and SMI sensors described herein.

At operation 706, the method 700 may include receiving at a control circuit or processor, from the set of one or more SMI sensors 704 and in response to the emission of the set of one or more beams of light, a set of one or more SMI signals.

At operation 716, the method 700 may include determining, based at least in part on the set of one or more SMI signals, a respiration characteristic of a user. As part of determining the respiration characteristic, and at operation 708, the method 700 may include performing an SMI signal analysis using the set of one or more SMI signals. The operation(s) at operation 708 may include, for example, generating (at operation 710) a velocity signal using one or more SMI signals. Determining the velocity signal may include applying filtering, performing an FFT, performing peak finding, and/or performing other types of signal processing on the received SMI signals (or on an average SMI signal, or on each SMI signal in a selected subset of the SMI signals, or on one or more SMI signals that have had common mode noise removed therefrom as described herein).

In some embodiments, the velocity signal may be further processed to generate (at operation 712) a displacement signal, which may be a measure of the relative displacement of the skin surface or may be a representation of the relative displacement of the skin surface. In some cases, the displacement signal may be generated by performing a mathematical integration of the velocity signal. In other cases, the displacement signal may be generated from the velocity signal using another method. In still other cases, the displacement signal may be generated from the SMI signal (or other signals) using other methods. The displacement signal may be filtered and/or may be further processed to reduce noise or remove any of a variety of signal artifacts.

In further embodiments, the displacement signal may be processed to generate (at operation 714) a volume signal, which may be a representation of the change in volume of air in a user's lungs during respiration. In some cases, the volume signal may be generated by performing a mathematical integration of the displacement signal, or may be generated by otherwise processing the SMI signals, velocity signal, displacement signal, and/or a combination of these or other signals using other methods. The volume signal may be filtered and/or may be further processed to reduce noise or remove any of a variety of signal artifacts.

At operation 716, the method 700 may also include determining which of the velocity signal, displacement signal, or volume signal provides a highest (or particular) signal quality, and selecting such signal as the respiration signal that is used for determining a respiration characteristic of a user at operation 718. In some cases, more than one respiration signal may be used to determine one or multiple respiration characteristics, or one or more respiration signal(s) may be output as respiration characteristics.

In addition, operation 716 may include determining which SMI sensor(s) of a set of SMI sensors provides a highest (or particular) signal quality. By way of example, it may be determined that an SMI sensor that provides an SMI signal of the highest quality when the SMI sensor produces an SMI signal that, when processed at operation 708, provides a volume signal with the highest signal quality, relative to the respective volume signals of the remaining SMI sensors of the set of SMI sensors. Thus, determining the respiration signal may include selecting the volume signal of the second SMI signal to be the respiration signal.

In embodiments where one or more of the SMI sensors is associated with a beam steering component, determining the respiration signal may include determining the SMI sensor, and the configuration of the associated beam steering component, that provides the highest signal quality. The respiration signal may then be based on such SMI signal, and on the configuration of the beam steering component.

In some variations, determining the respiration signal may include performing additional signal processing operations. For instance, the received SMI signals may include motion artifacts that may not be substantially removed by operations 710, 712, 714, and/or other signal processing operations. As described herein, the optical sensor subsystem 702 may include one or more SMI sensors of the set of SMI sensors 704 configured to generate SMI signals from other areas of the face or head. Such SMI sensors may generate SMI signals that may also contain the motion artifact, but may not contain signal components associated with respiration. Accordingly, SMI signals generated by these SMI sensors may be used to remove motion artifacts from an SMI signal that includes both the motion artifact and the respiration signal. As one example, determining the respiration signal at operation 716 may include receiving volume signals that include signal components associated with both respiration and motion artifacts, and receiving volume signals that include signal components associated primarily with motion artifacts. The volume signals that primarily contain motion artifact components may be removed from the volume signals that contain both respiration and motion artifact components.

At operation 718, the respiration characteristic may include, for example, a breathing condition or breathing pattern and/or the determined respiration signal. Examples of breathing conditions or breathing patterns may include breathing pattern disorder (BPD), dyspnea, orthopnea, apnea, Cheyne-Stokes respiration, bradypnea, tachypnea, hyperpnca, hyperventilation, hypoventilation, asthma, or changes in respiration volume or rate. Further, identifying breathing conditions may include identifying varying levels of nasal congestion, respiration congestion, and/or other conditions that may impair breathing.

In some variations, operation 718 may include determining and storing data associated with any of the identified breathing patterns and/or conditions. For instance, operation 718 may include determining a respiration rate associated with the breathing condition or pattern, estimating breathing volume, and/or determining other information associated with the identified breathing. For example, operation 718 may include determining the length of time, number of occurrences, and/or other metrics associated with regular or irregular breathing patterns or conditions.

In some variations, operation 718 may include determining additional conditions associated with use of the head-mountable frame. For example, operation 718 may include identifying an on-head condition of the head-mountable frame, and using the condition to enable and/or perform an operation of the head-mountable frame. In further examples, operation 718 may provide information about the quality of fitment of the head-mountable frame to the head of the user (e.g., position, tightness, slippage, etc.). Identifying the on-head condition and/or fitment of the head-mountable frame may include analyzing the respiration signal and/or the output from operation 708. For instance, the presence or absence of the respiration signal velocity signal, displacement signal, and/or volume signal, and/or the signal quality of these or other signals, may be used to identify the on-head condition and/or fitment of the head-mountable frame.

At operation 720, the method 700 may include providing respiration information to the user. For example, the respiration information described herein may be provided to a display of the head-mountable frame. In one instance, the respiration signal, or a respiration waveform indicative of the respiration signal may be provided to the display, such as may be part of a controlled breathing exercise, or for other purposes. In cases where an urgent breathing condition is identified, operation 720 may include providing an alert to the user, such as a visual, auditory, haptic, and/or other type of alert. In other embodiments, other respiration characteristics may be conveyed to the user-via text, graphics, audio, haptic notifications, or other means.

In some embodiments, operation 720 may include storing the respiration information, which may enable the user to access the respiration information at a later time, track breathing conditions, provide the respiration information to a healthcare provider, and/or use the respiration information for other purposes.

As may be appreciated, the method 700 may be performed on an ongoing basis, during a period in which the head-mountable frame is worn by the user. For example, method 700 may be continually performed, such that a highest quality respiration signal may be determined in real-time, or near real-time, and respiration information obtained. The method 700 may alternatively be formed when a software application (e.g., an “app”) is selected or activated within a graphical user interface (GUI).

The foregoing description, for purposes of explanation, uses specific nomenclature to provide a thorough understanding of the described embodiments. However, it will be apparent to one skilled in the art, after reading this description, that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art, after reading this description, that many modifications and variations are possible in view of the above teachings.

As described above, one aspect of the present technology may be the gathering and use of data available from various sources, including respiration information of a user. The present disclosure contemplates that, in some instances, this gathered data may be personal information that uniquely identifies or can be used to identify, locate, contact, or classify a specific person. Personal information can include, for example, data that is linked to respiration data or other personal information of a user (e.g., demographic data, location-based data, telephone numbers, email addresses, home addresses, data or records relating to a user's health or level of fitness (e.g., vital signs measurements, medication information, exercise information), date of birth, or any other identifying or personal information).

The present disclosure recognizes that the use of such personal information, in the present technology, can be used to the benefit of users. For example, the personal information can be used to authenticate a user to access their device, or gather performance metrics for the user's interaction with an augmented or virtual world. Further, other uses for personal information that benefit the user are also contemplated by the present disclosure. For instance, health and fitness data may be used to provide insights into a user's general wellness, or may be used as positive feedback to individuals using technology to pursue wellness goals.

The present disclosure contemplates that the entities responsible for the collection, analysis, disclosure, transfer, storage, or other use of such personal information will comply with well-established privacy policies and/or privacy practices. In particular, such entities should implement and consistently use privacy policies and practices that are generally recognized as meeting or exceeding industry or governmental requirements for maintaining personal information private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information and ensuring that others with access to the personal information adhere to their privacy policies and procedures. Further, such entities can subject themselves to evaluation by third parties to certify their adherence to widely accepted privacy policies and practices. In addition, policies and practices should be adapted for the particular types of personal information being collected and/or accessed and adapted to applicable laws and standards, including jurisdiction-specific considerations. For instance, in the US, collection of or access to certain health data may be governed by federal and/or state laws, such as the Health Insurance Portability and Accountability Act (HIPAA); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information. That is, the present disclosure contemplates that hardware and/or software elements can be provided to prevent or block access to such personal information. For example, in the case of advertisement delivery services, the present technology can be configured to allow users to select to “opt in” or “opt out” of participation in the collection of personal information during registration for services or anytime thereafter. In another example, users can select not to provide data to targeted content delivery services. In yet another example, users can select to limit the length of time data is maintained or entirely prohibit the development of a baseline profile for the user. In addition to providing “opt in” and “opt out” options, the present disclosure contemplates providing notifications relating to the access or use of personal information. For instance, a user may be notified upon downloading an app that their personal information will be accessed and then reminded again just before personal information is accessed by the app.

Moreover, it is the intent of the present disclosure that personal information should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data at a city level rather than at an address level), controlling how data is stored (e.g., aggregating data across users), and/or other methods.

Therefore, although the present disclosure broadly covers use of personal information to implement one or more various disclosed embodiments, the present disclosure also contemplates that the various embodiments can also be implemented without the need for accessing such personal information. That is, the various embodiments of the present technology are not rendered inoperable due to the lack of all or a portion of such personal information. For example, content can be selected and delivered to users by inferring preferences based on non-personal information or a bare minimum amount of personal information, such as the content being requested by the device associated with a user, other non-personal information available to the content delivery services, or publicly available information.

What is claimed is:

1. A device, comprising:
a head-mountable frame;
an optical sensor subsystem mounted to the head-mountable frame and including a set of one or more self-mixing interferometry (SMI) sensors, each SMI sensor of the set of one or more SMI sensors positioned on the head-mountable frame to be spaced a respective distance from a skin surface of a user when the head-mountable frame is worn by the user, and the set of one or more SMI sensors configured to emit a set of one or more beams of light that at least partially reflects from human skin; and
a processor configured to:
operate the optical sensor subsystem to cause the set of one or more SMI sensors to emit the set of one or more beams of light toward the skin surface of the user;
receive from the set of one or more SMI sensors, in response to the emission of the set of one or more beams of light, a set of one or more SMI signals; and
determine, based at least in part on the set of one or more SMI signals, a respiration characteristic of the user.

2. The device of claim 1, wherein at least one SMI sensor of the set of one or more SMI sensors is positioned on the head-mountable frame to direct a beam of light of the set of one or more beams of light toward a nose of the user.

3. The device of claim 1, wherein:
the optical sensor subsystem comprises a collimator positioned in an optical path of at least one SMI sensor of the set of one or more SMI sensors; and
the processor is configured to operate the optical sensor subsystem to cause the at least one SMI sensor to emit a beam of light of the set of one or more beams of light through the collimator and toward the skin surface of the user.

4. The device of claim 1, wherein the head-mountable frame is a glasses frame.

5. The device of claim 1, wherein the head-mountable frame is a headset.

6. The device of claim 1, wherein:
the processor is configured to identify a subset of one or more SMI sensors, of the set of one or more SMI sensors, that provides a subset of one or more SMI signals having a particular signal quality; and
the set of one or more SMI signals on which the respiration characteristic is based includes only the subset of one or more SMI signals provided by the subset of one or more SMI sensors.

7. The device of claim 1, wherein the processor is configured to:
perform at least one integration of at least one SMI signal of the set of one or more SMI signals, to generate at least one of a displacement signal or a volume signal; and
determine the respiration characteristic based at least in part on the displacement signal or the volume signal.

8. The device of claim 1, wherein the optical sensor subsystem comprises a beam steering component associated with at least one beam of light of the set of one or more beams of light.

9. The device of claim 8, wherein the processor is configured to:
operate the optical sensor subsystem to cause the beam steering component to sweep a beam of light, of the at least one beam of light, over a region of the skin surface of the user;
determine, based at least in part on an SMI signal associated with the beam of light, a particular orientation of the beam of light with respect to the region of the skin surface;
operate the optical sensor subsystem to cause the beam steering component to direct the beam of light toward the skin surface in the particular orientation; and
determine the respiration characteristic based at least in part on the SMI signal associated with the beam of light.

10. A method for determining a respiration characteristic, comprising:
operating an optical sensor subsystem to cause a set of one or more non-contact self-mixing interferometry (SMI) sensors in the optical sensor subsystem to emit a set of one or more beams of light toward a skin surface of a user;
receiving a set of one or more SMI signals from the set of one or more non-contact SMI sensors;
performing at least one integration of each of the received set of one or more SMI signals to generate a set of one or more integrated SMI signals; and
determining the respiration characteristic based on a subset of one or more integrated SMI signals of the set of one or more integrated SMI signals.

11. The method of claim 10, wherein at least one non-contact SMI sensor of the set of one or more non-contact SMI sensors is positioned to direct a beam of light of the set of one or more beams of light toward a nose of the user.

12. The method of claim 10, wherein at least one non-contact SMI sensor of the set of one or more non-contact SMI sensors is positioned to direct a beam of light of the set of one or more beams of light toward a portion of the skin surface adjacent an upper lip of the user.

13. The method of claim 10, wherein at least one beam of light of the set of one or more beams of light is emitted through a collimator.

14. The method of claim 10, wherein the determined respiration characteristic comprises a respiration rate of the user.

15. The method of claim 10, wherein the determined respiration characteristic comprises a breathing pattern associated with irregular breathing.

16. A device, comprising:
a head-mountable frame;
an optical sensor subsystem mounted to the head-mountable frame and including,
a set of one or more self-mixing interferometry (SMI) sensors, each SMI sensor of the set of one or more SMI sensors positioned on the head-mountable frame to be spaced a respective distance from a skin surface of a user when the head-mountable frame is worn by the user; and
a collimator positioned in an optical path of at least one SMI sensor in the set of one or more SMI sensors; and
a processor configured to:
operate the optical sensor subsystem to obtain a set of one or more SMI signals from the set of one or more SMI sensors; and
determine a respiration characteristic of the user based at least in part on the set of one or more SMI signals.

17. The device of claim 16, wherein the processor is configured to operate the device as an augmented reality (AR) head-mounted device or a virtual reality (VR) head-mounted device.

18. The device of claim 16, wherein the respiration characteristic is a respiration rate.

19. The device of claim 16, wherein the respiration characteristic is a breathing pattern.

20. The device of claim 16, wherein the processor is configured to select a subset of one or more SMI sensors, of the set of one or more SMI sensors, that provides a subset of one or more SMI signals, of the set of one or more SMI signals, wherein the respiration characteristic is based at least in part on the subset of one or more SMI signals.
